(19) United States
(12) Patent Application Publication
(10) Pub. No.: US 2025/0002505 A1
(43) Pub. Date: Jan. 2, 2025

(54) SYNTHESIS OF MACROYCLIC COMPOUNDS

(71) Applicant: TURNING POINT THERAPEUTICS, INC., San Diego, CA (US)

(72) Inventors: Biman PAL, San Diego, CA (US); Jing LIU, San Diego, CA (US); Kevin MARTIN, San Diego, CA (US); Han ZHANG, San Diego, CA (US)

(21) Appl. No.: 18/698,692

(22) PCT Filed: Sep. 30, 2022
(86) PCT No.: PCT/US2022/077376

§ 371 Date: Apr. 4, 2024

Related U.S. Application Data
(60) Provisional application No. 63/252,227, filed on Oct. 5, 2021.

(51) Int. Cl. C07D 498/22; C07D 498/14

(52) U.S. Cl. CPC C07D 498/22; C07D 498/14

(57) ABSTRACT

The present disclosure provides new methods of preparing a compound of Formula (I). Such methods can prepare the compound at any scale, including gram and kilogram scale.

### BACKGROUND

Protein kinases regulate various functions in the cell including cell growth, proliferation and survival. Dysregulation of protein kinases is often the cause of many solid malignancies. The use of protein kinase inhibitors has led to substantial clinical benefit in patients harboring oncogenic aberrations. Protein kinase inhibitors have been approved for clinical treatment of cancers. RET is a receptor tyrosine kinase that is expressed with its highest levels in early embryogenesis (during which it has diverse roles in different tissues) and decreases to relatively low levels in normal adult tissues (Pachnis, V., et al. Development 1993, 119, 1005-1017). RET activation regulates the downstream signalling pathways (RAS/MAPK/ERK, PI3K/AKT, and JAK-STAT etc.), leading to cellular proliferation, migration, and differentiation (Mulligan, LM. Nat Rev Cancer. 2014, 14(3):173-86).

Gain-of-function mutations of RET with constitutive activation have been found in heritable and sporadic tumors including activating point mutations within the full-length RET protein or genomic rearrangements that produce chimeric RET oncoproteins in the cytosol. The heritable oncogenic RET mutations are found in multiple endocrine neoplasia type 2 (MEN2) including medullary thyroid cancer (MTC) and familial MTC with more than 80 pathogenic variants spanning RET exons 5-16 reported (Mulligan, LM. Nat Rev Cancer. 2014, 14(3):173-86).

Macrocyclic RET inhibitors have been reported in the art. For example, Compound 5 of U.S. Pat. No. 10,745,416, referred to herein as the compound of Formula (I), was shown to have RET IC₅₀ of 1 nM in a biochemical kinase assay. Crystalline forms of the compound of Formula (I) have been described in PCT publication WO 2020/257169.

There is a need for improved methods of preparing macrocyclic RET inhibitors, such as the compound of Formula (I).

### BRIEF SUMMARY

The present disclosure is directed to methods of preparing a compound of Formula (I). Provided herein is a method of preparing a compound of Formula (I):

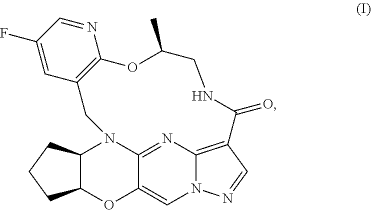 [SMILES: C[C@H]1CNC(=O)c2cnn3cc4c(nc23)N(Cc2cc(F)cnc2O1)[C@@H]1CCC[C@@H]1O4]

or a pharmaceutically acceptable salt, solvate or hydrate thereof, comprising:
(a) combining a compound of Formula (II):

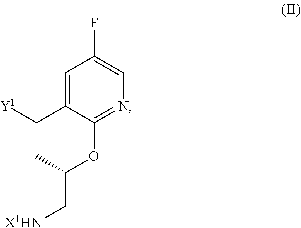

wherein X¹ is a nitrogen protecting group, and Y¹ is a leaving group,
and a compound of Formula (III):

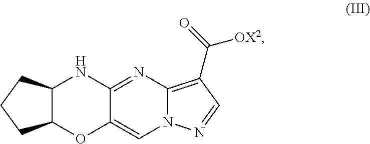

wherein X² is an oxygen protecting group, to form a compound of Formula (IV):

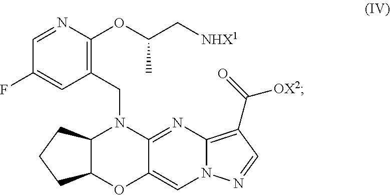

(b) deprotecting the compound of Formula (IV) to form a compound of Formula (V):

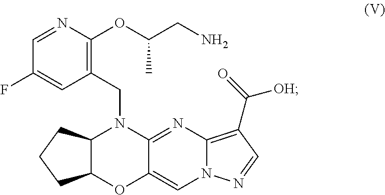 [SMILES: C[C@@H](CN)Oc1ncc(F)cc1CN1c2nc3c(C(=O)O)cnn3cc2O[C@H]2CCC[C@H]21]

and
(c) cyclizing the compound of Formula (V), thereby preparing the compound of Formula (I).

Further provided herein is a method of preparing a compound of Formula (I):

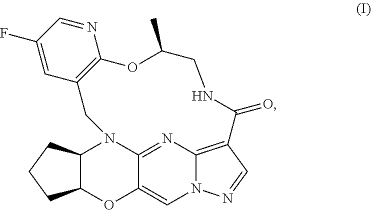 [SMILES: C[C@H]1CNC(=O)c2cnn3cc4c(nc23)N(Cc2cc(F)cnc2O1)[C@@H]1CCC[C@@H]1O4]

or a pharmaceutically acceptable salt, solvate or hydrate thereof, comprising: cyclizing a compound of Formula (VI):

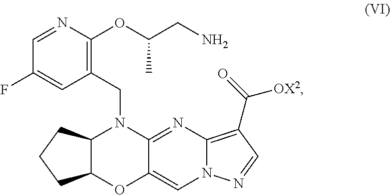

wherein X² is an oxygen protecting group, to form a compound of Formula (I).

Also provided is a compound of Formula (VI), or a salt, hydrate, or solvate thereof:

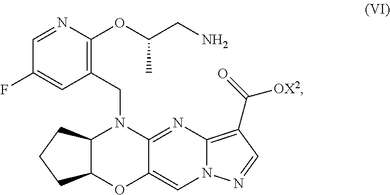

wherein X² is C₁-C₆ alkyl, C₆-C₁₀ aryl, or C₇-C₁₂ alkylenearyl, wherein the aryl or alkylenearyl is optionally substituted with halogen, C₁-C₆ alkyl, or C₁-C₆ alkoxy.

### DETAILED DESCRIPTION

The present disclosure provides methods of preparing a compound of Formula (I):

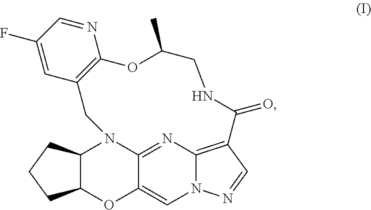 [SMILES: C[C@H]1CNC(=O)c2cnn3cc4c(nc23)N(Cc2cc(F)cnc2O1)[C@@H]1CCC[C@@H]1O4]

or a pharmaceutically acceptable salt, solvate or hydrate thereof. The methods described herein are capable of generating the compound of Formula (I) at any scale, for example, from 1 gram to 1 kg.

The disclosure further provides new intermediates useful for preparing the compound of Formula (I), such as a compound of Formula (VI), or a salt, hydrate, or salt thereof.

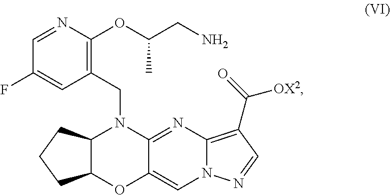

wherein X² is C₁-C₆ alkyl, C₆-C₁₀ aryl, or C₇-C₁₂ alkylenearyl, wherein the aryl or alkylenearyl is optionally substituted with halogen, C₁-C₆ alkyl, or C₁-C₆ alkoxy.

In the following description, certain specific details are set forth in order to provide a thorough understanding of various embodiments of the invention. However, one skilled in the art will understand that the invention may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### I. DEFINITIONS

Unless the context requires otherwise, throughout the present specification and claims, the word “comprise” and variations thereof, such as, “comprises” and “comprising” are to be construed in an open, inclusive sense, that is as “including, but not limited to”.

Reference to “about” a value or parameter herein includes (and describes) embodiments that are directed to that value or parameter per se. For example, description referring to “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Alkyl” is a linear or branched saturated monovalent hydrocarbon. For example, an alkyl group can have 1 to 18 carbon atoms (i.e., C₁₋₁₈ alkyl) or 1 to 8 carbon atoms (i.e., C₁₋₈ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include heptyl, octyl, nonyl, decyl, undecyl, dodecyl, pentadcyl, hexadecyl, heptadecyl and octadecyl.

“Alkylene” is a linear or branched saturated divalent hydrocarbon. For example, an alkylene group can have 1 to 18 carbon atoms (i.e., C₁₋₁₈ alkylene) or 1 to 8 carbon atoms (i.e., C₁₋₈ alkylene) or 1 to 6 carbon atoms (i.e., C₁₋₆ alkylene) or 1 to 4 carbon atoms (i.e., C₁₋₄ alkylene). Examples of alkylene groups include, but are not limited to, methylene (—CH₂—), ethylene (—CH₂CH₂—), 1-propylene (—CH₂CH₂CH₂—), 2-propylene (—CH(CH₃)—CH₂—), 1-butylene (—CH₂CH₂CH₂CH₂—), 2-methyl-1-propylene (—CH₂CH(CH₃)CH₂—), 2-butylene (—CH(CH₃)CH₂CH₂—), 1-pentylene (—CH₂CH₂CH₂CH₂CH₂—), and 2-pentylene (—CH(CH₃)CH₂CH₂CH₂—). The alkylene groups can be further substituted with a variety of substituents described within. Alkylene groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxyalkyl” refers an alkoxy group linked to an alkyl group which is linked to the remainder of the compound such that the alkyl group is divalent. Alkoxyalkyl can have any suitable number of carbon, such as from 2 to 6 (C₂₋₆ alkoxyalkyl), 2 to 5 (C₂₋₅ alkoxyalkyl), 2 to 4 (C₂₋₄ alkoxyalkyl), or 2 to 3 (C₂₋₃ alkoxyalkyl). The number of carbons refers to the total number of carbons in the alkoxy and the alkyl group. For example, C₆ alkoxyalkyl refers to ethoxy (C₂ alkoxy) linked to a butyl (C₄ alkyl), and n-propoxy (C₃ alkoxy) linked to a isopropyl (C₃ alkyl). Alkoxy and alkyl are as defined above where the alkyl is divalent, and can include, but is not limited to, methoxymethyl (CH₃OCH₂—), methoxyethyl (CH₃OCH₂CH₂—) and others.

“Halo” or “halogen” as used herein refers to fluoro (—F), chloro (—Cl), bromo (—Br) and iodo (—I).

“Haloalkyl” as used herein refers to an alkyl as defined herein, wherein one or more hydrogen atoms of the alkyl are independently replaced by a halo substituent, which may be the same or different. For example, C₁₋₄ haloalkyl is a C₁₋₄ alkyl wherein one or more of the hydrogen atoms of the C₁₋₄ alkyl have been replaced by a halo substituent. Examples of haloalkyl groups include but are not limited to fluoromethyl, fluorochloromethyl, difluoromethyl, difluorochloromethyl, trifluoromethyl, 1,1,1-trifluoroethyl and pentafluoroethyl.

“Aryl” as used herein refers to a single all carbon aromatic ring or a multiple condensed all carbon ring system wherein at least one of the rings is aromatic. For example, in some embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having 9 to 20 carbon atoms, e.g., 9 to 16 carbon atoms, in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1,2,3,4-tetrahydronaphthyl. Non-limiting examples of aryl groups include, but are not limited to, phenyl, indenyl, naphthyl, 1,2,3,4-tetrahydronaphthyl, anthracenyl, and the like.

“Alkylenearyl” as used herein refers to an alkylene as defined herein, wherein the alkylene is independently substituted by one or more aryl substituents, which may be the same or different. For example, C₇₋₁₂ alkylenearyl includes a C₁ alkylene attached to a C₆ phenyl ring and a C₂ alkylene attached to a C₁₀ naphthyl ring. Examples of alkylenearyl groups include but are not limited to benzyl, phenethyl, and 1-methylnaphthyl. The alkylenearyl groups can be further substituted with a variety of substituents described within. Alkylenearyl groups can be substituted or unsubstituted.

A “protecting group” is used to mask the reactivity of a given group, e.g., an oxygen or a nitrogen, during one or more chemical reactions, and revealed at a later stage upon deprotection. Protecting groups are available, commonly known and used, and are optionally used to prevent side reactions with the protected group during synthetic procedures, i.e. routes or methods to prepare a compound of the present disclosure. For the most part the decision as to which groups to protect, when to do so, and the nature of the chemical protecting group will be dependent upon the chemistry of the reaction to be protected against (e.g., acidic, basic, oxidative, reductive or other conditions) and the intended direction of the synthesis. The protecting groups do not need to be, and generally are not, the same if the compound is substituted with multiple protecting groups. In general, protecting groups will be used to protect functional groups such as carboxyl, hydroxyl, thio, or amino groups and to thus prevent side reactions or to otherwise facilitate the synthetic efficiency. The order of deprotection to yield free, deprotected groups is dependent upon the intended direction of the synthesis and the reaction conditions to be encountered, and may occur in any order as determined by the artisan. Exemplary oxygen protecting groups and nitrogen protecting groups and their corresponding chemical cleavage reactions are described in Protective Groups in Organic Synthesis, Theodora W. Greene and Peter G. M. Wuts (John Wiley & Sons, Inc., New York, 1999, ISBN 0-471-16019-9.) See also Kocienski, Philip J.; Protecting Groups (Georg Thieme Verlag Stuttgart, New York, 1994.)

The invention disclosed herein is also meant to encompass all pharmaceutically acceptable compounds of Formula I being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹C, ¹³N, ¹⁵N, ¹⁵O, ¹⁷O, ¹⁸O, ³¹P, ³²p, ³⁵S, ¹⁸F, ³⁶C, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labeled compounds of Formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I can generally be prepared by conventional techniques known to those skilled in the art or by processes analogous to those described in the Examples as set out below using an appropriate isotopically-labeled reagent in place of the non-labeled reagent previously employed.

### II. METHODS OF PREPARING

Provided herein is a method of preparing a compound of Formula (I):

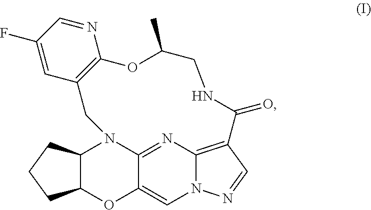 [SMILES: C[C@H]1CNC(=O)c2cnn3cc4c(nc23)N(Cc2cc(F)cnc2O1)[C@@H]1CCC[C@@H]1O4]

or a pharmaceutically acceptable salt, solvate or hydrate thereof, comprising:
(a) combining a compound of Formula (II):

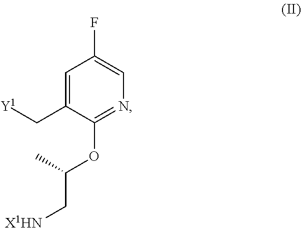

wherein
X¹ is a nitrogen protecting group, and
Y¹ is a leaving group,
and a compound of Formula (III):

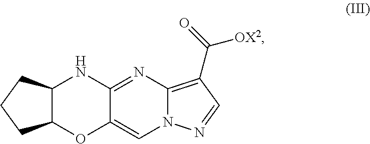

wherein X² is an oxygen protecting group,
to form a compound of Formula (IV):

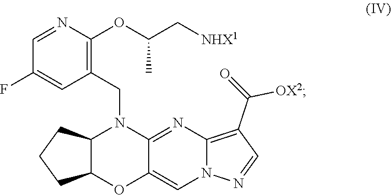

(b) deprotecting the compound of Formula (IV) to form a compound of Formula (V):

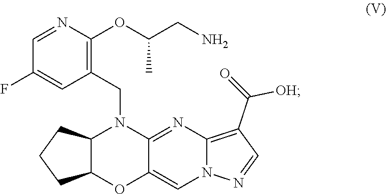 [SMILES: C[C@@H](CN)Oc1ncc(F)cc1CN1c2nc3c(C(=O)O)cnn3cc2O[C@H]2CCC[C@H]21]

and
(c) cyclizing the compound of Formula (V), thereby preparing the compound of Formula (I).

Any suitable X¹ nitrogen protecting group can be used in the method for preparing the compound of Formula (I). In some embodiments, X¹ is phthaloyl (Pht), 4-methoxybenzyloxycarbonyl, o-nitrophenylsulfenyl (Nps), p-toluenesulfonyl (Ts), 3,5-dimethoxybenzyloxycarbonyl, 2-nitro-4,5-dimethoxybenzyloxycarbonyl (NVOC), 2-(4-biphenyl)isopropoxycarbonyl (Bpoc), α,α-dimethyl-3,5-dimethoxybenzyloxycarbonyl (Ddz), tetrachlorophthaloyl (TCP), tert-butoxycarbonyl (Boc), carboxybenzoyl (Cbz), 9-fluorenylmethyloxy carbonyl (Fmoc), allyloxy carbonyl (Aloc), or trityl (Tr). For example, X¹ can be tert-butoxycarbonyl (Boc).

Any suitable Y¹ leaving group can be used in the method for preparing the compound of Formula (I). In some embodiments, Y¹ is chloride, bromide, iodide, methanesulfonate (OMs), benzenesulfonate (OBs), toluenesulfonate (OTs), or trifluoromethanesulfonate (OTf). For example, Y¹ can be methanesulfonate (OMs).

In some embodiments of the method, the combining further comprises a first base. The first base can be an inorganic base. Any suitable inorganic base can be used in the method of preparing a compound of Formula (I). Exemplary inorganic bases include lithium bicarbonate, sodium bicarbonate, potassium bicarbonate, cesium bicarbonate, lithium carbonate, sodium carbonate, potassium carbonate, cesium carbonate, lithium hydroxide, sodium hydroxide, potassium hydroxide, and cesium hydroxide. In some embodiments, the inorganic base is lithium carbonate, sodium carbonate, potassium carbonate, or cesium carbonate. For example, the inorganic base can be cesium carbonate.

In some embodiments of the method, the compound of Formula (II) has the structure:

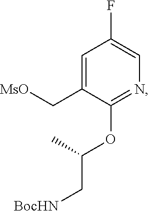 [SMILES: C[C@@H](CNC(=O)OC(C)(C)C)Oc1ncc(F)cc1COS(C)(=O)=O]

or a salt thereof.

In some embodiments of the method, the compound of Formula (III) has the structure:

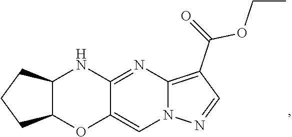 [SMILES: CCOC(=O)c1cnn2cc3c(nc12)N[C@@H]1CCC[C@@H]1O3]

or a salt thereof.

In some embodiments of the method, the compound of Formula (IV) has the structure:

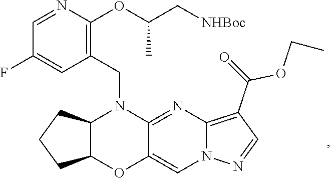 [SMILES: CCOC(=O)c1cnn2cc3c(nc12)N(Cc1cc(F)cnc1O[C@@H](C)CNC(=O)OC(C)(C)C)[C@@H]1CCC[C@@H]1O3]

or a salt thereof.

The method of combining a compound of Formula (II) and a compound of Formula (III) to form a compound of Formula (IV) can be performed in any suitable temperature and solvent known in the art. In some embodiments, the temperature is from about 0° C. to about 100° C., such as from about 0° C. to about 50° C., from about 10° C. to about 40° C., from about 10° C. to about 30° C., from about 20° C. to about 30° C., or from about 20° C. to about 25° C. For example, the temperature of combining a compound of Formula (II) and a compound of Formula (III) can be from about 10° C. to about 30° C. In another example, the temperature of combining a compound of Formula (II) and a compound of Formula (III) can be from about 20° C. to about 25° C.

Suitable solvents for the method of combining a compound of Formula (II) and a compound of Formula (III) to form a compound of Formula (IV) include dichloromethane (DCM), dioxane, tetrahydrofuran (THF), 2-methyltetrahydrofuran (2-Me-THF), dimethylformamide (DMF), dimethylacetamide (DMA), and N-methylpyrrolidone (NMP), or any combination thereof. In some embodiments, the solvent for combining a compound of Formula (II) and a compound of Formula (III) comprises dimethylformamide (DMF). For example, the solvent for combining a compound of Formula (II) and a compound of Formula (III) can be 2-methyltetrahydrofuran (2-Me-THF) and dimethylformamide (DMF).

The method of deprotecting a compound of Formula (IV) to form a compound of Formula (V) can comprise one or more steps. In some embodiments, deprotecting a compound of Formula (IV) comprises preparing a compound of Formula (VI):

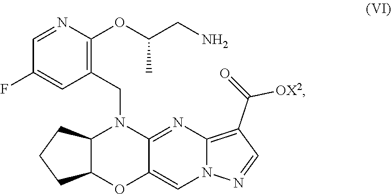

wherein X² is an oxygen protecting group.

In some embodiments, deprotecting a compound of Formula (IV) comprises preparing a compound of Formula (VII):

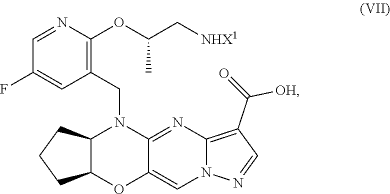

wherein X¹ is a nitrogen protecting group.

The method of deprotecting a compound of Formula (IV) to form a compound of Formula (V) can be performed in any suitable temperature and solvent known in the art. In some embodiments, the temperature is from about 0° C. to about 100° C., such as from about 0° C. to about 50° C., from about 10° C. to about 40° C., from about 10° C. to about 30° C., from about 20° C. to about 30° C., or from about 20° C. to about 25° C. For example, the temperature of deprotecting a compound of Formula (IV) to form a compound of Formula (V) can be from about 10° C. to about 30° C. In another example, the temperature of deprotecting a compound of Formula (IV) to form a compound of Formula (V) can be from about 20° C. to about 25° C.

Suitable solvents for the method of deprotecting a compound of Formula (IV) to form a compound of Formula (V) include dichloromethane (DCM), ethanol, water, dioxane, tetrahydrofuran (THF), 2-methyltetrahydrofuran (2-Me-THF), dimethylformamide (DMF), dimethylacetamide (DMA), and N-methylpyrrolidone (NMP), or any combination thereof. For example, the solvent can be ethanol and water. In another example, the solvent can be dichloromethane (DCM) and dioxane.

In some embodiments of the method, the cyclizing a compound of Formula (V) comprises a coupling agent. Any suitable coupling agent can be used in the method for preparing a compound of Formula (I). In some embodiments, the cyclizing comprises pentafluorophenyl diphenylphosphinate (FDPP).

In some embodiments of the method, the cyclizing a compound of Formula (V) comprises a base. Any suitable inorganic or organic base can be used in cyclizing a compound of Formula (V). In some embodiments, the base is an organic base. Suitable organic bases include trialkylammonium bases, such as trimethylamine, trimethylamine, and diisopropylethylamine, as well as pyridine bases, such as pyridine, 2-methylpyridine, and 4-dimethylaminopyridine (DMAP). In some embodiments of the method, the cyclizing a compound of Formula (V) comprises triethylamine.

The method of cyclizing a compound of Formula (V) to form a compound of Formula (I) can be performed in any suitable temperature and solvent known in the art. In some embodiments, the temperature is from about 0° C. to about 100° C., such as from about 0° C. to about 50° C., from about 10° C. to about 40° C., from about 10° C. to about 30° C., from about 20° C. to about 30° C., or from about 20° C. to about 25° C. For example, the temperature of cyclizing a compound of Formula (V) can be from about 10° C. to about 30° C. In another example, the temperature of cyclizing a compound of Formula (V) can be from about 20° C. to about 25° C.

Suitable solvents for the method of cyclizing a compound of Formula (V) to form a compound of Formula (I) include dichloromethane (DCM), ethanol, water, dioxane, tetrahydrofuran (THF), 2-methyltetrahydrofuran (2-Me-THF), dimethylformamide (DMF), dimethylacetamide (DMA), and N-methylpyrrolidone (NMP), or any combination thereof. For example, the solvent for cyclizing a compound of Formula (V) can be dichloromethane (DCM).

In some embodiments, the method further comprises deprotecting the compound of Formula (IV) to prepare a compound of Formula (VI):

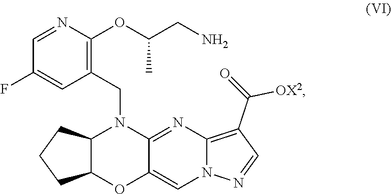

wherein X² is an oxygen protecting group,
and combining the compound of Formula (VI) and a second base to form the compound of Formula (V):

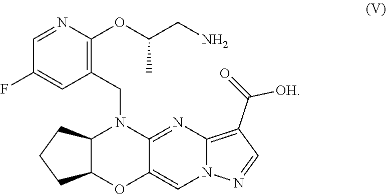 [SMILES: C[C@@H](CN)Oc1ncc(F)cc1CN1c2nc3c(C(=O)O)cnn3cc2O[C@H]2CCC[C@H]21]

Any suitable second base can be used in the method in combining the compound of Formula (VI) to form the compound of Formula (V). In some embodiments, the second base is an inorganic base. Exemplary inorganic bases include lithium bicarbonate, sodium bicarbonate, potassium bicarbonate, cesium bicarbonate, lithium carbonate, sodium carbonate, potassium carbonate, cesium carbonate, lithium hydroxide, sodium hydroxide, potassium hydroxide, and cesium hydroxide. In some embodiments, the second base is sodium hydroxide.

The method of deprotecting the compound of Formula (IV) to prepare a compound of Formula (VI) can be performed in any suitable temperature and solvent known in the art. In some embodiments, the temperature is from about 0° C. to about 100° C., such as from about 0° C. to about 50° C., from about 10° C. to about 40° C., from about 10° C. to about 30° C., from about 20° C. to about 30° C., or from about 20° C. to about 25° C. For example, the temperature of deprotecting the compound of Formula (IV) to prepare a compound of Formula (VI) can be from about 10° C. to about 30° C. In another example, the temperature of deprotecting the compound of Formula (IV) to prepare a compound of Formula (VI) can be from about 20° C. to about 25° C.

The method of combining the compound of Formula (VI) to form the compound of Formula (V) can be performed in any suitable temperature and solvent known in the art. In some embodiments, the temperature is from about 0° C. to about 100° C., such as from about 0° C. to about 50° C., from about 10° C. to about 40° C., from about 10° C. to about 30° C., from about 20° C. to about 30° C., or from about 20° C. to about 25° C. For example, the temperature of combining the compound of Formula (VI) to form the compound of Formula (V) can be from about 10° C. to about 30° C. In another example, the temperature of combining the compound of Formula (VI) to form the compound of Formula (V) can be from about 20° C. to about 25° C.

Suitable solvents for the method of combining the compound of Formula (VI) to form the compound of Formula (V) include dichloromethane (DCM), ethanol, water, dioxane, tetrahydrofuran (THF), 2-methyltetrahydrofuran (2-Me-THF), dimethylformamide (DMF), dimethylacetamide (DMA), and N-methylpyrrolidone (NMP), or any combination thereof. For example, the solvent can be ethanol and water.

In some embodiments, the method of preparing a compound of Formula (I) or a pharmaceutically acceptable salt, solvate or hydrate thereof, comprises:
(a) combining a compound of Formula (II) having structure:

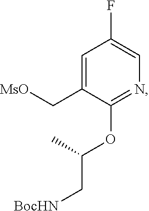 [SMILES: C[C@@H](CNC(=O)OC(C)(C)C)Oc1ncc(F)cc1COS(C)(=O)=O]

or a salt thereof,
   a compound of Formula (III) having structure:

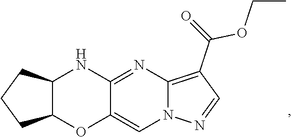 [SMILES: CCOC(=O)c1cnn2cc3c(nc12)N[C@@H]1CCC[C@@H]1O3]

or a salt thereof, and cesium carbonate,
to form a compound of Formula (IV) having structure:

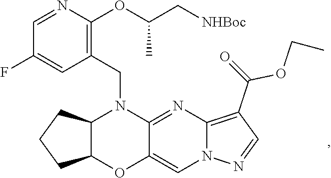 [SMILES: CCOC(=O)c1cnn2cc3c(nc12)N(Cc1cc(F)cnc1O[C@@H](C)CNC(=O)OC(C)(C)C)[C@@H]1CCC[C@@H]1O3]

or a salt thereof,
(b) (i) deprotecting the compound of Formula (IV) to prepare a compound of Formula (VI) having structure:

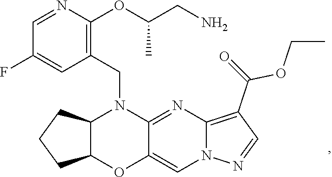 [SMILES: CCOC(=O)c1cnn2cc3c(nc12)N(Cc1cc(F)cnc1O[C@@H](C)CN)[C@@H]1CCC[C@@H]1O3]

or a salt thereof,
(ii) combining the compound of Formula (VI) and sodium hydroxide to form the compound of Formula (V):

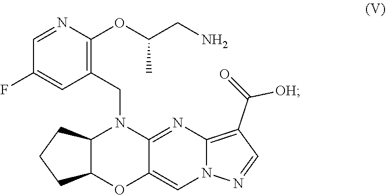 [SMILES: C[C@@H](CN)Oc1ncc(F)cc1CN1c2nc3c(C(=O)O)cnn3cc2O[C@H]2CCC[C@H]21]

and
(c) cyclizing the compound of Formula (V) with pentafluorophenyl diphenylphosphinate (FDPP), thereby preparing the compound of Formula (I).

Further provided herein is a method of preparing a compound of Formula (I):

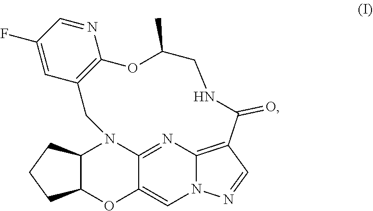 [SMILES: C[C@H]1CNC(=O)c2cnn3cc4c(nc23)N(Cc2cc(F)cnc2O1)[C@@H]1CCC[C@@H]1O4]

or a pharmaceutically acceptable salt, solvate or hydrate thereof, comprising:
cyclizing a compound of Formula (VI):

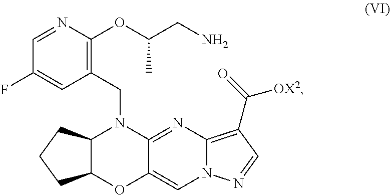

wherein X² is an oxygen protecting group,
to form a compound of Formula (I).

Any suitable X² oxygen protecting group can be used in the method for preparing the compound of Formula (I) described herein. In some embodiments, X² is C₁-C₆ alkyl, C₆-C₁₀ aryl, or C₇-C₁₂ alkylenearyl, wherein the aryl or alkylenearyl is optionally substituted with halogen, C₁-C₆ alkyl, or C₁-C₆ alkoxy. In some embodiments, X² is C₁-C₆ alkyl. For example, X² can be ethyl.

The cyclizing of a compound of Formula (VI) can further comprise an acid or a base capable of effecting the intramolecular cyclization. Any suitable acid or base can be used in cyclizing the compound of Formula (VI), and can be either inorganic or organic. In some embodiments, the cyclizing comprises lithium amide, lithium ethoxide, 1,5,7-triazabicyclo[4.4.0]dec-5-ene, potassium carbonate, or acetic acid. For example, the cyclizing can comprise 1,5,7-triazabicyclo[4.4.0]dec-5-ene.

The method of cyclizing the compound of Formula (VI) to form the compound of Formula (I) can be performed in any suitable temperature and solvent known in the art. In some embodiments, the temperature is from about 0° C. to about 200° C., such as from about 50° C. to about 200° C., from about 50° C. to about 150° C., from about 100° C. to about 200° C., from about 100° C. to about 150° C., or from about 125° C. to about 135° C. For example, the temperature of cyclizing the compound of Formula (VI) to form the compound of Formula (I) can be from about 100° C. to about 150° C. In another example, the temperature of cyclizing the compound of Formula (VI) to form the compound of Formula (I) can be from about 125° C. to about 135° C.

Suitable solvents for the method of cyclizing the compound of Formula (VI) to form the compound of Formula (I) include dichloromethane (DCM), ethanol, water, dioxane, tetrahydrofuran (THF), 2-methyltetrahydrofuran (2-Me-THF), dimethylformamide (DMF), dimethylacetamide (DMA), and N-methylpyrrolidone (NMP), or any combination thereof, or can be performed neat. In some embodiments, the method of cyclizing the compound of Formula (VI) to form the compound of Formula (I) is performed neat.

In some embodiments, the method of preparing a compound of Formula (I) or a pharmaceutically acceptable salt, solvate or hydrate thereof, comprises cyclizing a compound of Formula (VI) having structure:

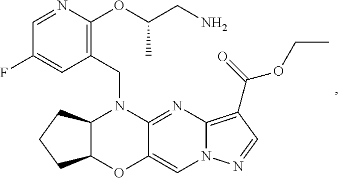 [SMILES: CCOC(=O)c1cnn2cc3c(nc12)N(Cc1cc(F)cnc1O[C@@H](C)CN)[C@@H]1CCC[C@@H]1O3]

or a salt thereof, with 1,5,7-triazabicyclo[4.4.0]dec-5-ene to form a compound of Formula (I).

A method of the present disclosure can prepare a crystalline form of Formula (I). Accordingly, in some embodiments of the method described herein, a compound of Formula (I) is a crystalline form of Formula (I). Suitable crystalline forms of Formula (I) have been described in PCT publication WO 2020/257169, which is hereby incorporated in its entirety. In some embodiments, the crystalline form of Formula (I) is a hydrate. In some embodiments, the crystalline form of Formula (I) has a powder X-ray diffraction pattern comprising a peak at diffraction angle (2θ) of 20.1±0.1. In some embodiments, the crystalline form of Formula (I) has a powder X-ray diffraction pattern comprising peaks at diffraction angles (2θ) of 11.3±0.1 and 20.1±0.1. In some embodiments, the crystalline form of Formula (I) has a powder X-ray diffraction pattern comprising peaks at diffraction angles (2θ) of 11.3±0.1, 20.1±0.1, and 23.9±0.1. In some embodiments, the crystalline form of Formula (I) has a powder X-ray diffraction pattern comprising peaks at diffraction angles (2θ) of 11.3±0.1, 18.0±0.1, 20.1±0.1, and 23.9±0.1. In some embodiments, the crystalline form of Formula (I) has a powder X-ray diffraction pattern comprising peaks at diffraction angles (2θ) of 10.3±0.1, 11.3±0.1, 18.0±0.1, 20.1±0.1, and 23.9±0.1. In some embodiments, the crystalline form of Formula (I) has a powder X-ray diffraction pattern comprising peaks at diffraction angles (2θ) of 10.3±0.1, 11.3±0.1, 16.6±0.1, 18.0±0.1, 20.1±0.1, and 23.9±0.1. In some embodiments, the crystalline form of Formula (I) has a powder X-ray diffraction pattern comprising peaks at diffraction angles (2θ) of 10.3±0.1, 11.3±0.1, 16.6±0.1, 18.0±0.1, 20.1±0.1, 20.5±0.1, and 23.9±0.1. In some embodiments, the crystalline form of Formula (I) has a differential scanning calorimetry (DSC) thermogram having an endotherm at from about 150° C. to about 160° C. In some embodiments, the crystalline form of Formula (I) has a differential scanning calorimetry (DSC) thermogram having an endotherm at about 156° C.

In some embodiments of the method, the crystalline form of Formula (I) is anhydrous. In some embodiments, the crystalline form of Formula (I) has a powder X-ray diffraction pattern comprising a peak at diffraction angle (2θ) of 18.8±0.1. In some embodiments, the crystalline form of Formula (I) has a powder X-ray diffraction pattern comprising peaks at diffraction angles (2θ) of 18.8±0.1 and 20.9±0.1. In some embodiments, the crystalline form of Formula (I) has a powder X-ray diffraction pattern comprising peaks at diffraction angles (2θ) of 12.3±0.1, 18.8±0.1, and 20.9±0.1. In some embodiments, the crystalline form of Formula (I) has a powder X-ray diffraction pattern comprising peaks at diffraction angles (2θ) of 12.3±0.1, 18.8±0.1, 19.4±0.1, and 20.9±0.1. In some embodiments, the crystalline form of Formula (I) has a powder X-ray diffraction pattern comprising peaks at diffraction angles (2θ) of 12.3±0.1, 13.1±0.1, 18.8±0.1, 19.4±0.1, and 20.9±0.1. In some embodiments, the crystalline form of Formula (I) has a powder X-ray diffraction pattern comprising peaks at diffraction angles (2θ) of 12.3±0.1, 13.1±0.1, 15.3±0.1, 18.8±0.1, 19.4±0.1, and 20.9±0.1. In some embodiments, the crystalline form of Formula (I) has a powder X-ray diffraction pattern comprising peaks at diffraction angles (2θ) of 9.4±0.1, 12.3±0.1, 13.1±0.1, 15.3±0.1, 18.8±0.1, 19.4±0.1, and 20.9±0.1. In some embodiments, the crystalline form of Formula (I) has a differential scanning calorimetry (DSC) thermogram having an endotherm at from about 235° C. to about 245° C. In some embodiments, the crystalline form of Formula (I) has a differential scanning calorimetry (DSC) thermogram having an endotherm at about 240° C.

Further provided herein is a compound of Formula (VI), or a salt, hydrate, or solvate thereof:

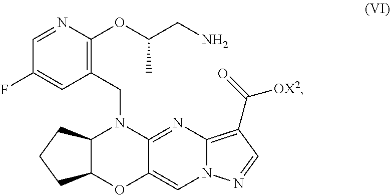

wherein X² is C₁-C₆ alkyl, C₆-C₁₀ aryl, or C₇-C₁₂ alkylenearyl, wherein the aryl or alkylenearyl is optionally substituted with halogen, C₁-C₆ alkyl, or C₁-C₆ alkoxy. In some embodiments, X² is C₁-C₆ alkyl. For example, X² can be ethyl.

In some embodiments, the compound of Formula (VI) has the structure:

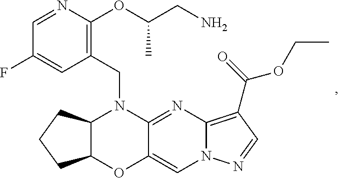 [SMILES: CCOC(=O)c1cnn2cc3c(nc12)N(Cc1cc(F)cnc1O[C@@H](C)CN)[C@@H]1CCC[C@@H]1O3]

or a salt thereof.

### III. EXAMPLES

Exemplary methods of the description will now be described by reference to the specific examples that follow. Artisans will recognize that, to obtain the various compounds herein, starting materials may be suitably selected so that the ultimately desired substituents will be carried through the reaction scheme with or without protection as appropriate to yield the desired product.

### Abbreviations

| | |
|---|---|
| ° C. | degrees Celsius |
| 2-Me—THF | 2-methyltetrahydrofuran |
| Boc | tert-butoxycarbonyl |
| Cbz | carboxybenzoyl |
| DCM | dichloromethane |
| DMF | dimethylformamide |
| eq | equivalents |
| FDPP | pentafluorophenyl diphenylphosphinate |
| g | grams |
| h | hours |
| HPLC | high performance liquid chromatography |
| kg | kilograms |
| min | minutes |
| mmol | millimoles |
| Ms | methanesulfonyl |
| MsCl | methanesulfonyl chloride |
| TBD | 1,5,7-triazabicyclodec-5-ene |
| TBME | tert-butyl methyl ether |
| TEA | triethylamine |
| TFA | trifluoroacetic acid |
| THF | tetrahydrofuran |

### Example 1. Preparation of Intermediate Compound 1-4

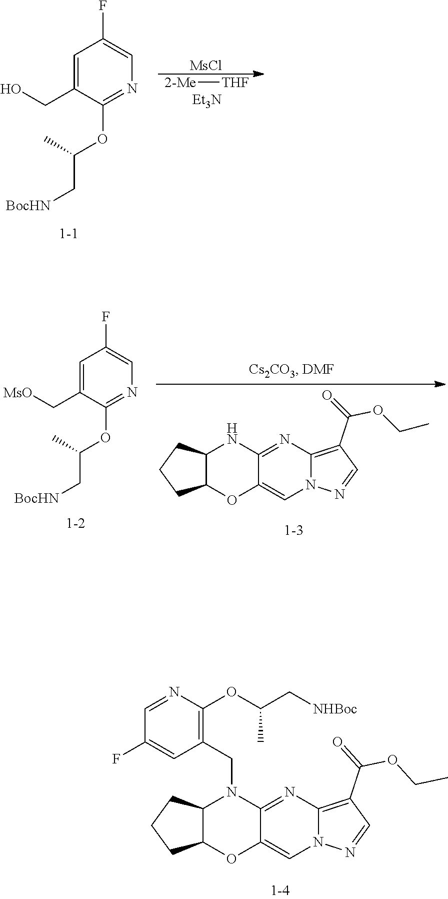

A solution of Compound 1-1 (26.0 kg) in 2-Me-THF (41 kg) was charged to the reactor, followed by a 2-Me-THF (55 kg) rinse. At 20-25° C. to the reactor was charged triethylamine (8.4 kg) followed by 2-Me-THF (13 kg) rinse. The reactor was cooled to 0-5° C. and methanesulfonyl chloride (8.9 kg) was charged over 35 min while maintaining the batch at 0° C. to 10° C. A 2-Me-THF (7 kg) rinse was performed. The batch was warmed up to 20-25° C. over 30 min, and then was stirred for an additional 50 min. The waste solids were removed by filtration and the filter cake was washed with 2-Me-THF (28 kg) to give a solution of Compound 1-2.

To another reactor was charged Compound 1-3 (1 eq., 16.0 kg), micronized cesium carbonate (45.2 kg), and DMF (45 kg). The resulting slurry was stirred at 20-25° C. for 85 min. The Compound 1-2 solution was transferred to the slurry over 4.25 h while maintaining the batch at 20-25° C. A 2-Me-THF (13 kg) rinse was performed. The batch was stirred at 20-25° C. for 4 h, whereupon water (128 kg) was added. After 30 min of stirring the layers were separated. To the reactor was added water (128 kg) and 2-Me-THF (28 kg) and after 35 min of stirring, the layers were again separated. The combined organics batch was distilled under reduced pressure to a volume of about 60 L. TBME (95 kg) was added and the organics batch was distilled under reduced pressure to a volume of 60 L. The addition of TBME and distillation was repeated. To the batch at 20-25° C. was added n-heptane (55 kg) over 1 hour and the batch was stirred for 1.5 h. The solid product was collected by filtration and the filter cake was washed with pre-mixed TBME:n-heptane (13 kg:20 kg). The resulting Compound 1-4 was dried on the filter under a nitrogen stream (27.6 kg, 87% yield). HPLC purity was 97.5%.

### Example 2. Formula (I) Preparation by Deprotection and Coupling

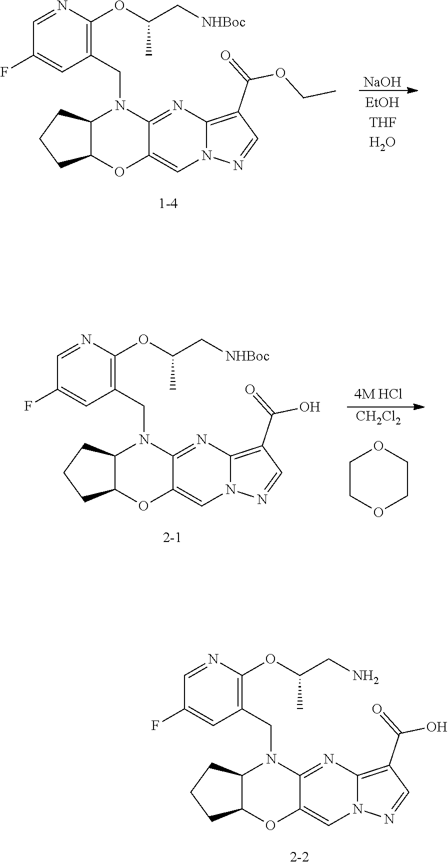

Compound 1-4 (27.4 kg) and ethanol (87.1 kg) were charged at room temperature to a glass lined reactor, under nitrogen. With moderate agitation the contents were stirred at about 20° C. for 15 min. With vigorous agitation, sodium hydroxide 50% solution (12.0 kg) was charged over 30 min while maintaining the batch at 20-25° C., followed by a rinse with water (96 L). The batch was warmed up to 70-75° C. over 69 min while stirring. The batch was stirred vigorously at 70-75° C. for an additional 5 h 45 min. The reaction mixture was distilled under reduced pressure to a volume of 110-125 L. With moderate agitation the batch was cooled to 10-15° C. To reaction mixture with vigorous agitation was added hydrochloric acid, 37% w/w (17.0 kg) and the batch was allowed to exotherm to 10-25° C., followed by a rinse with water (28 L). To the reaction mixture was added methylene chloride (292.2 kg) and after 40 min of stirring, agitation was stopped and the mixture was allowed to stand for 35 min before separating the layers. The lower organic layer was transferred to a second reactor (˜225 L). The organic layer was washed twice with water (165 L) at room temperature for 20 min. Methylene chloride (146.0 kg) was added at room temperature, and the organics were distilled under vacuum to a volume of 110-125 L. Additional methylene chloride was added (65.6 kg).

The reaction mixture was cooled to 10-15° C. With vigorous agitation was added 4M HCl in dioxane (51.2 kg) over 4 h while maintaining the temperature at 10-30° C., followed by a rinse with methylene chloride (35.9 kg). The batch was maintained at 21-23° C. for 18 h, and then was cooled to 0-5° C. With vigorous agitation was added water (28 kg) over 50 min maintaining the temperature at 3-6° C., followed by sodium hydroxide, 50% solution (20.5 kg) over 1 h maintaining the temperature at 4-13° C. The reaction mixture was allowed to warm to 23° C. for 1 h 45 min. With vigorous agitation was added hydrochloric acid, 37% (9.76 kg) over 70 min maintaining the temperature at 20-25° C., followed by a rinse with water (3.0 kg). After 50 min of stirring, to the reaction was added sodium hydroxide, 50% solution (0.2 kg) in one portion. With vigorous agitation the batch was maintained at room temperature for 3.5 h, and then filtered. The resulting filter cake was washed with toluene (96.4 kg). The product was dried on the filter under a nitrogen stream, then dried at 50-55° C. in a vacuum oven for 2 days. The amount of intermediate Compound 2-2 was 16.2 kg (76% yield).

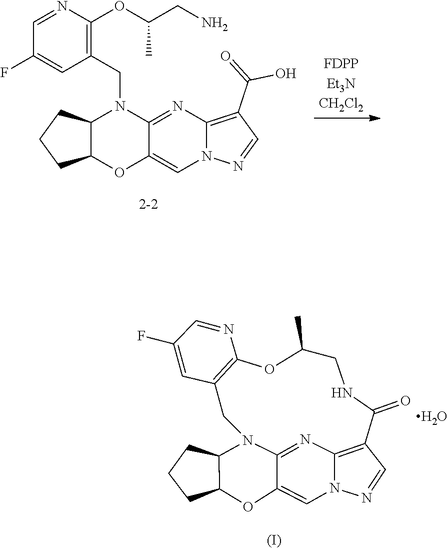

To a stainless steel Reactor 1 at 20-25° C. was charged Compound 2-2 (23.5 kg, 16.0 kg dry basis, 1.0 eq.), DCM (148 kg), and triethylamine (11 kg, 3 eq.) followed by a rinse with DCM (21 kg).

To Reactor 2 at 20-25° C. was charged DCM (212 kg). About 15 L of the starting material DCM solution from Reactor 1 was transferred into a GR (glass receiver), then transferred over to the DCM in Reactor 2. To Reactor 2 was charged via the split butterfly valve (SBV) system at 20-25° C., FDPP (1.53 kg, about 10% of the total amount) and the batch in Reactor 2 was stirred at 20-25° C. for 30 min. The portion wise addition from Reactor 1 into Reactor 2, followed by the FDPP addition was repeated another 9 times (total 10 additions). After the last addition, Reactor 1 and the GR were rinsed with DCM (21 kg).

To Reactor 2 was charged, via SBV, Celite (0.8 kg) and the batch was stirred at 20-25° C. for 1 hour. The slurry from Reactor 2 was filtered, followed by a DCM (42 kg) rinse. A solution of sodium carbonate (11.5 kg) in distilled deionized (DD) water (120 kg) was charged to the reaction mixture over 1 h, followed by a water rinse (8 kg) and the batch was stirred at 20-25° C. for 30 min. The layers were separated and the lower (organic layer) was transferred to another reactor.

To the organic layer was charged water (128 kg) and the batch was stirred at 20-25° C. for 20 min. The layers were separated and water wash was repeated. The organic layer was separated, charged DCM (85 kg), and the batch was distilled at atmospheric pressure to a volume of 145 L (target 144-160 L). Ethanol was added, the mixture filtered, followed by a rinse forward with DCM (42 kg). The batch was distilled under vacuum to a volume of 60 L (target volume 56-67 L). To the batch was charged ethanol (189 kg) and the batch was distilled under reduced pressure to a volume of 75 L (target volume 72-80 L).

The batch was warmed to an internal temperature of 46° C. and water (80 kg) was charged in small portions over 1 h 20 min at 45-50° C. The batch was cooled at 40-45° C. and was stirred vigorously for 1 h 15 min, while monitoring for crystallization. With vigorous stirring, the batch was cooled and was held at 20-25° C. for 4 hours. The solid product was collected by filtration and the filter cake was washed with a pre-mixed mixture of ethanol (13 kg) and water (16 kg). The compound of Formula (I) was dried on the filter under a nitrogen stream for 2.5 days, and then was packaged as a crystalline hydrate (13.9 kg, 91% yield).

### Example 3. Formula (I) Preparation with Separate Deprotection

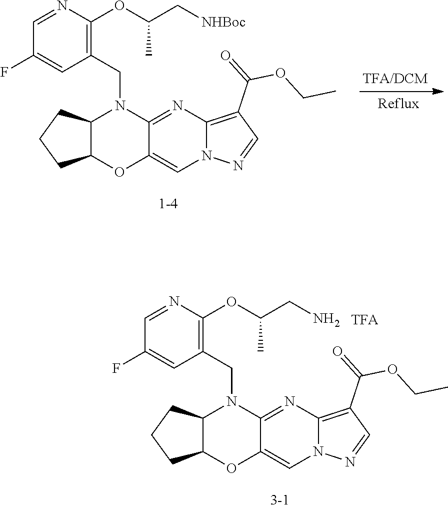 [SMILES: CCOC(=O)c1cnn2cc3c(nc12)N(Cc1cc(F)cnc1O[C@@H](C)CNC(=O)OC(C)(C)C)[C@@H]1CCC[C@@H]1O3 ; CCOC(=O)c1cnn2cc3c(nc12)N(Cc1cc(F)cnc1O[C@@H](C)CN)[C@@H]1CCC[C@@H]1O3]

To the reactor was charged, via split butterfly valve (SBV) and glove bag, 31.9 kg of Compound 1-4. Under nitrogen, methylene chloride (170 kg) was charged and the mixture was stirred vigorously at 20-25° C. for 50 min. Trifluoroacetic acid (TFA) (44.7 kg) was charged at 21-23° C. over 35 min. followed by a DCM rinse (43 kg). The mixture was heated to reflux for 13.5 h. The batch temperature was adjusted to 20-25° C. and ethyl alcohol (103 kg) was charged with vigorous agitation. The mixture was heated to reflux and the solvent was distilled at atmospheric pressure to a target volume of 155-194 L over about 3 h. The batch temperature was adjusted to 20-25° C. and ethyl alcohol (252 kg) was charged with moderate agitation. The solvent was distilled under vacuum to a target volume of 155-194 L at a temperature range of from 21° C. to 32° C. The resulting mixture was heated to 40-45° C. for 2 h, then cooled to 0-5° C. over 3.5 h, and stirred at 0-5° C. for 2-3 h. The product was collected by filtration, and the filter cake washed with ethyl alcohol (38 kg). The product was dried by passing a stream of nitrogen for 2 days (26.2 kg, 80% yield).

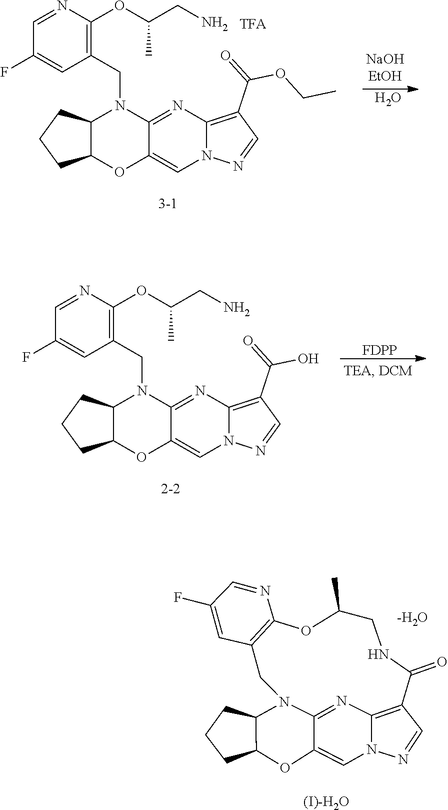

Compound 3-1 (19.9 kg), ethanol (95.2 kg) and water (60.2 kg) were charged at ambient temperature to a glass lined reactor, under nitrogen. With vigorous agitation the contents were stirred at 22-25° C. for 30 min. With vigorous agitation, sodium hydroxide 50% solution (8.2 kg) was charged over 30 min. while maintaining the batch at 21 to 23° C., followed by a rinse with water (40 L). The batch was warmed up to 70° C. over 90 min. The batch was stirred vigorously at 70-74° C. for an additional 7 h. The batch was cooled to 22-23° C. and was transferred, followed by a rinse with premixed ethanol (8 kg) and water (10.2 kg).

In a separate reactor was charged 31% w/w hydrochloric acid (12.1 kg) and water (8 kg) to make a 6N HCl solution. To the reaction mixture was charged DCM (398 kg), followed by 15.9 kg of the 6N HCl solution and the batch was stirred vigorously for 18 min at 21° C., while maintaining the temperature during the addition was 21 to 24° C. The layers were allowed to separate, and the lower organic layer was transferred (˜320 L). The organic layer was twice washed with a solution of sodium chloride (6.0 kg) in water (54 kg). The batch was distilled at atmospheric pressure to a target volume of 160-180 L. DCM (212 kg) was added, and the batch was distilled at atmospheric pressure to a target volume of 160-180 L. The DCM addition and distillation was repeated. Triethylamine (10.5 kg) and DCM (27 kg) were added to give a solution of Compound 2-2.

To a reactor was charged DCM (264 kg) at 22° C. About 20 L of the Compound 2-2 DCM solution (ca. 10% by volume of the batch) was added to the DCM in the reactor. To the reactor was charged via the SBV system at 20-25° C. solid FDPP (1.71 kg, about 10% of the total amount) and the reaction mixture was stirred at 20-25° C. for 1.5 h. The portion wise addition of Compound 2-2, followed by the solid FDPP addition was repeated another 9 times (total of 10 additions). The stirring time in between the FDPP additions ranged from 35 min to 2 hours. After the last addition, DCM (27 kg) was added, and the batch was stirred vigorously for 3 hours, whereupon a solution of sodium carbonate (10.9 kg) in water (100 kg) was added.

The layers in the reaction mixture were separated and the lower organic layer (400 L) was washed with water (2×100 kg), and then distilled at atmospheric pressure to a target volume 180-200 L. The batch was cooled to 23° C. and was transferred, followed by a rinse with DCM (27 kg). The batch was distilled at atmospheric pressure to a volume of 110 L (target 90-110 L). The batch was cooled to 25° C. and ethyl alcohol (237 kg) was added. The batch was distilled under vacuum to a target volume 80-90 L. The product started crystallizing out.

The batch was warmed to an internal temperature of 46° C. and water (50 kg) was added in portions. With vigorous stirring, the batch was cooled at 24° C. over 2 hours and was held at 22-24° C. for 5 hours. The solid product was collected by filtration and the filter cake was washed with stirring with a pre-mixed mixture of ethanol (12 kg) and water (15 kg). The cake was dried under vacuum to give the Formula (I) as a hydrate: 11.03 kg (73% yield).

### Example 4. Formula (I) Preparation by Cyclization of Amino Ester

### Screening of Reagents for Amino Ester Cyclization

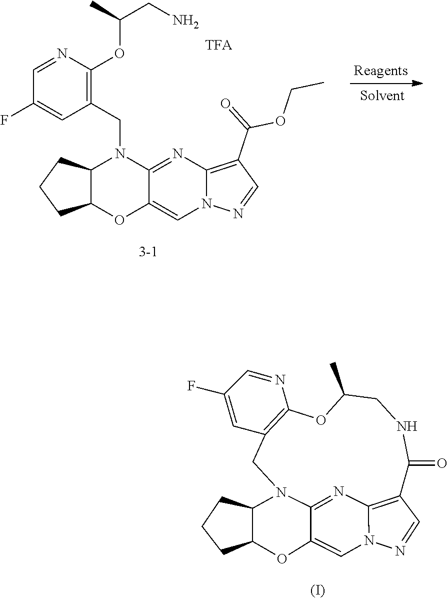

A variety of reagents and solvents were screened at small scale to evaluate the cyclization of Compound 3-1 to Formula (I) under different conditions. The reactions were performed with Compound 3-1 ranging from 50 to 200 mg scale.

The results are summarized in the table below.

| No. | Reagents | Solvent | Temp/Time | Yield (HPLC) |
|---|---|---|---|---|
| 1 | LiNH₂ (1.2 eq.) | THF (10 vol) | 80° C./32 h | 41% |
| 2 | C₂H₅OLi (1.5 eq.) | Toluene (10 vol) | 110° C./32 h | 24% |
| 3 | C₂H₅OLi (1.5 eq.) | Chlorobenzene | 130° C./32 h | 79% |
| | | (10 vol) | | |
| 4 | DABAL—Me₃ (1.2 eq.) | Toluene (10 vol) | 110° C./24 h | 44% |
| 5 | 3-Nitrophenol (0.5 eq.)/ | Chlorobenzene | 130° C./48 h | 6% |
| | K₂CO₃ THF (0.5 eq.) | (20 vol) | | |
| 6 | Trifluoroethanol (0.5 eq.)/ | 1,4-Dioxane (20 vol) | 120° C./32 h | 7% |
| | K₂CO₃ (0.5 eq.) | | | |
| 7 | Acetic acid (0.5 eq.) | Toluene (20 vol) | 110° C./16 h | 44% |
| 8 | t-BuOK (2.0 eq.) | Chlorobenzene | 130° C./24 h | No product |
| | | (20 vol) | | |
| 9 | Lithium hexamethyldisilazide | Chlorobenzene | 50° C./16 h | Degradation |
| | (2.5 eq.) | (20 vol) | | (no product) |
| 10 | Lithium chloride (1.5 eq.) | Chlorobenzene | 130° C./32 h | No Product |
| | | (20 vol) | | |

Gram-Scale Amino Ester Cyclization with 1,5,7-Triazabicyclodec-5-Ene

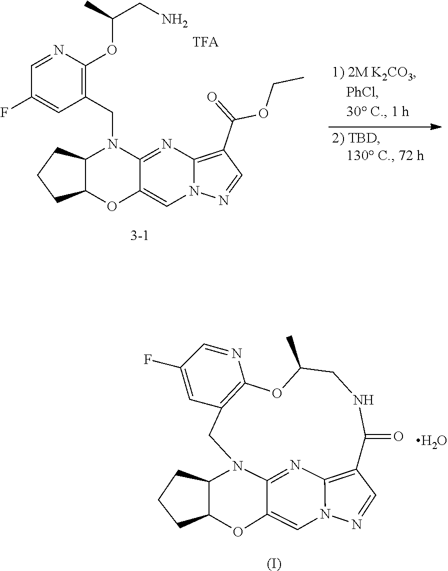

Cyclization of Compound 3-1 was accomplished in gram scale using 1,5,7-triazabicyclodec-5-ene (TBD). In a flask was added Compound 3-1 (10.0 g, 17.6 mmol, 1.0 eq.), chlorobenzene (200 mL, 20 vol) and 2M K₂CO₃ (18 mL, 2.0 eq.). The mixture was stirred at 30° C. for 1 h. The mixture was then transferred to a separatory funnel and the phases separated. The chlorobenzene layer was transferred to a sealed tube vessel.

1,5,7-Triazabicyclodec-5-ene (TBD) (3.7 g, 26.4 mmol, 1.5 eq.) was added to the chlorobenzene layer in the sealed tube vessel indicated above, and the vessel sealed. The solution was heated at 130° C. for 72 h. The mixture was allowed to cool. The reaction was quenched with 1M HCl (100 mL) to pH 2 followed by the addition of ethyl acetate (300 mL). The biphasic mixture was transferred to a separatory funnel and the phases separated. The aqueous layer was further extracted with ethyl acetate (100 mL×2). The combined organic layers were washed sequentially with saturated sodium bicarbonate solution (300 mL), water (300 mL) and brine (300 mL), and then dried over anhydrous Na₂SO₄. The mixture was filtered and the filtrate was concentrated under vacuum at 45° C., followed by chasing with ethyl acetate (2×50 mL) to give light colored yellow solid. The solid was dried under vacuum at 50° C. to give Formula (I) (6.8 g). HPLC purity was 90.4% at 235 nm.

To the crude Formula (I) (6.8 g) was added ethyl acetate-heptane (306 mL; 2:1) and the mixture heated to 95° C. To the solution was added silica gel (1.7 g, 0.25 eq.) to the mixture and stirred at 95° C. for 1 h. The suspension was cooled to 20° C. and filtered after 2 h. The filtrate was concentrated under vacuum and the resulting residue dried under vacuum. The purification process was repeated.

The resulting solid was dissolved in ethanol at 40-45° C. and water (31 mL) added to the ethanolic solution at 40-45° C. over 1 h. The resulting mixture was vigorously stirred for 1 h. The resulting slurry was cooled to 20-25° C. and stirred for 4 h. The mixture was filtered. The filter cake was washed with aqueous ethanol (22 mL; 1:1.2), and dried under vacuum at 55° C. to give 4.2 g of Formula (I) crystalline hydrate as a white solid (98.8% by HPLC at 235 nm, 56% yield).

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of preparing a compound of Formula (I): 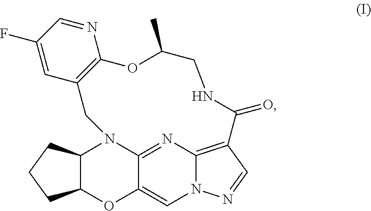 [SMILES: C[C@H]1CNC(=O)c2cnn3cc4c(nc23)N(Cc2cc(F)cnc2O1)[C@@H]1CCC[C@@H]1O4]
or a pharmaceutically acceptable salt, solvate or hydrate thereof, comprising:
(a) combining a compound of Formula (II): 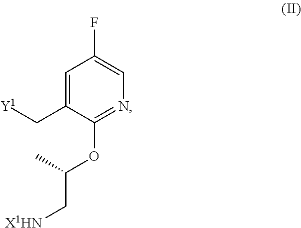
wherein
X¹ is a nitrogen protecting group, and
Y¹ is a leaving group,
and a compound of Formula (III): 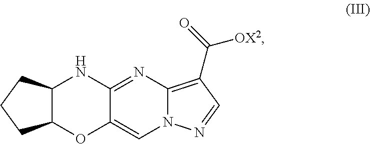
wherein X² is an oxygen protecting group,
to form a compound of Formula (IV): 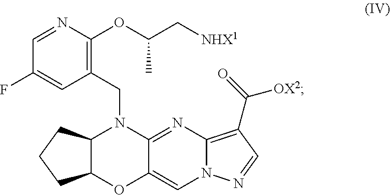
(b) deprotecting the compound of Formula (IV) to form a compound of Formula (V): 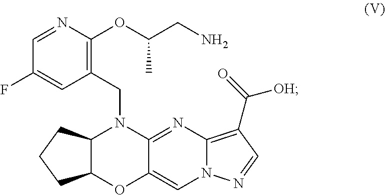 [SMILES: C[C@@H](CN)Oc1ncc(F)cc1CN1c2nc3c(C(=O)O)cnn3cc2O[C@H]2CCC[C@H]21]
and
(c) cyclizing the compound of Formula (V), thereby preparing the compound of Formula (I).

2. The method of claim 1, wherein X¹ is phthaloyl (Pht), 4-methoxybenzyloxycarbonyl, o-nitrophenylsulfenyl (Nps), p-toluenesulfonyl (Ts), 3,5-dimethoxybenzyloxycarbonyl, 2-nitro-4,5-dimethoxybenzyloxycarbonyl (NVOC), 2-(4-biphenyl)isopropoxycarbonyl (Bpoc), α,α-dimethyl-3,5-dimethoxybenzyloxycarbonyl (Ddz), tetrachlorophthaloyl (TCP), tert-butoxycarbonyl (Boc), carboxybenzoyl (Cbz), 9-fluorenylmethyloxy carbonyl (Fmoc), allyloxy carbonyl (Aloc), or trityl (Tr).

3. The method of claim 1, wherein X¹ is tert-butoxycarbonyl (Boc).

4. The method of claim 1, wherein X² is C₁-C₆ alkyl, C₆-C₁₀ aryl, or C₇-C₁₂ alkylenearyl, wherein the aryl or alkylenearyl is optionally substituted with halogen, C₁-C₆ alkyl, or C₁-C₆ alkoxy.

5. The method of claim 1, wherein X² is C₁-C₆ alkyl.

6. The method of claim 1, wherein X² is ethyl.

7. The method of claim 1, wherein Y¹ is chloride, bromide, iodide, methanesulfonate (OMs), benzenesulfonate (OBs), toluenesulfonate (OTs), or trifluoromethanesulfonate (OTf).

8. The method of claim 1, wherein Y¹ is methanesulfonate (OMs).

9. The method of claim 1, wherein the combining further comprises a first base.

10. The method of claim 9, wherein the first base comprises an inorganic base.

11. The method of claim 10, wherein the inorganic base is lithium carbonate, sodium carbonate, potassium carbonate, or cesium carbonate.

12. The method of claim 1, wherein the solvent for combining the compound of Formula (II) and the compound of Formula (III) comprises dimethylformamide (DMF).

13. The method of claim 1, wherein the temperature of combining the compound of Formula (II) and the compound of Formula (III) is from about 10° C. to about 30° C.

14. The method of claim 1, wherein the cyclizing comprises pentafluorophenyl diphenylphosphinate (FDPP).

15. The method of claim 1, wherein the solvent for cyclizing a compound of Formula (V) is dichloromethane (DCM).

16. (canceled)

17. The method of claim 1, further comprising deprotecting the compound of Formula (IV) to prepare a compound of Formula (VI): 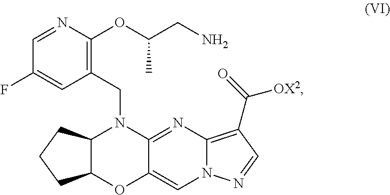
wherein X² is an oxygen protecting group,
and combining the compound of Formula (VI) and a second base to form the compound of Formula (V):

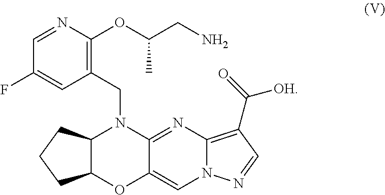 [SMILES: C[C@@H](CN)Oc1ncc(F)cc1CN1c2nc3c(C(=O)O)cnn3cc2O[C@H]2CCC[C@H]21]

18. (canceled)

19. A method of preparing a compound of Formula (I): 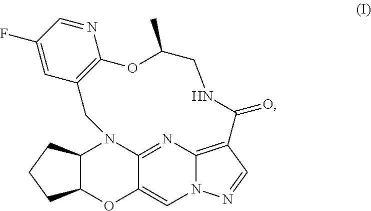 [SMILES: C[C@H]1CNC(=O)c2cnn3cc4c(nc23)N(Cc2cc(F)cnc2O1)[C@@H]1CCC[C@@H]1O4]
or a pharmaceutically acceptable salt, solvate or hydrate thereof, comprising:
cyclizing a compound of Formula (VI): 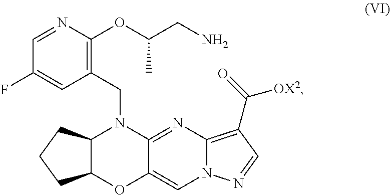
wherein X² is an oxygen protecting group,
to form a compound of Formula (I).

20. The method of claim 19, wherein the cyclizing comprises lithium amide, lithium ethoxide, 1,5,7-triazabicyclo[4.4.0]dec-5-ene, potassium carbonate, or acetic acid.

21. The method of claim 19, wherein the cyclizing comprises 1,5,7-triazabicyclo[4.4.0]dec-5-ene.

22.-23. (canceled)

24. A compound of Formula (VI), or a salt, hydrate, or solvate thereof: 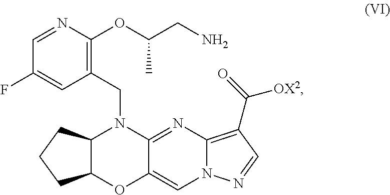
wherein X² is C₁-C₆ alkyl, C₆-C₁₀ aryl, or C₇-C₁₂ alkylenearyl, wherein the aryl or alkylenearyl is optionally substituted with halogen, C₁-C₆ alkyl, or C₁-C₆ alkoxy.

25. The compound of claim 24, having the structure: 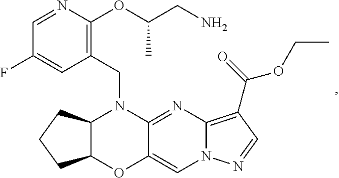 [SMILES: CCOC(=O)c1cnn2cc3c(nc12)N(Cc1cc(F)cnc1O[C@@H](C)CN)[C@@H]1CCC[C@@H]1O3]
or a salt thereof.
